(19) United States
(12) Patent Application Publication
(10) Pub. No.: US 2015/0119836 A1
(43) Pub. Date: Apr. 30, 2015

(54) Ostomy stoma waste overflow process and bag

(71) Applicants: Dale Martin Frimel, North Royalton, OH (US); Anita Marie Zindroski, Parma, OH (US)

(72) Inventors: Dale Martin Frimel, North Royalton, OH (US); Anita Marie Zindroski, Parma, OH (US)

(21) Appl. No.: 14/065,456

(22) Filed: Oct. 29, 2013

(51) Int. Cl. A61F 5/44; A61M 39/24

(52) U.S. Cl. CPC A61F 5/4405; A61M 39/24
USPC 604/335

(57) ABSTRACT

An ostomy stoma waste overflow control method and ostomy bag which allows human waste to flow from one ostomy bag to another ostomy bag via a one directional flow valve, which has the ability to automatically open when the stoma waste pressure within the first, or primary, bag reaches a pressure near, but under, the pressure that would cause the primary bag to leak stoma body waste onto the person wearing it. Present ostomy bags can leak onto the person wearing it if the bag's stoma waste pressure increases by over filling, or by being physically bumped or squeezed (such as when a person is sleeping on it). This new invention prevents an ostomy bag from breaking the ostomy seal (which seals the ostomy body flange to the person's skin) and leaking human waste material onto the person wearing it. Leaking can cause great embarrassment and pain since human waste will burn the skin where the ostomy flange is attached to the user. Leaking can also greatly stain the bed, sheets and blankets where a person is sleeping.

### CROSS REFERENCE TO RELATED APPLICATIONS

| | | | |
|---|---|---|---|
| U.S. Pat. No. 8,449,511 | Activity Ostomy Bag | Anderson | May 28, 2013 |
| U.S. Pat. No. 8,211,072 | Ostomy Bag | Smith | Jul. 3, 2012 |
| US PUB NO 2013005382 | Ostomy Devices | Maidl | Feb. 28, 2013 |
| U.S. Pat. No. 8,075,539 | Waste Management | Nishytala | Dec. 13, 2011 |
| | System | | |
| U.S. Pat. No. 7,918,836 | Ostomy Bag with Irrigation | Gill | Apr. 5, 2011 |
| U.S. Pat. No. 7,722,586 | Ostomy Appliance | Mallejans | May 25, 2010 |
| U.S. Pat. No. 7,722,585 | Pouch for Collecting | Falconer | May 25, 2010 |
| | Human Waste | | |
| U.S. Pat. No. 7,470,263 | Ostomy System | Strobech | Dec. 30, 2008 |
| U.S. Pat. No. 7,416,543 | Drainage Bag | Brown | Aug. 26, 2008 |
| U.S. Pat. No. 7,223,260 | Collecting Bag | Hansen | May 29, 2007 |
| U.S. Pat. No. 7,150,728 | Collecting Bag | Hansen | Dec. 19, 2006 |
| | having Vent | | |
| U.S. Pat. No. 7,001,367 | Valved Ostomy | Arkinstall | Feb. 21, 2006 |
| | Drainage Device | | |
| U.S. Pat. No. 6,902,551 | Ostomy Appliance | Hansen | Jun. 7, 2005 |
| U.S. Pat. No. 6,659,988 | Ostomy Appliance | Steer | Dec. 9, 2003 |
| U.S. Pat. No. 6,656,169 | Ostomy Pouch | Steer | Dec. 2, 2003 |
| U.S. Pat. No. 6,332,879 | Ostomy Appliance | Nielsen | Dec. 25, 2001 |
| U.S. Pat. No. 6,165,159 | Gas Vent for Ostomy Bag | Blauton | Dec. 26, 2000 |
| U.S. Pat. No. 5,658,267 | Colstomy Collection | Cofacello | Aug. 19, 1997 |
| | Pouch | | |

### Classification 604/332

### STATEMENT REGARDING FEDERALLY SPONSORED RESEARCH DEVELOPMENT

Not applicable.

### REFERENCE TO SEQUENCE LISTING, A TABLE, OR A COMPUTER PROGRAM LISTING COMPACT DISK APPENDIX

Not applicable.

### BACKGROUND OF THE INVENTION

A person who has had an ileostomy must use an external bag to collect their human waste material. This waste exits the body from internal organs through an opening in their abdomen called a stoma. Present art uses a latex-like bag that collects human waste material exiting from this stoma. The bag is attached via an ostomy body flange that is held on to the person's abdomen with an adhesive coated cloth surrounding the ostomy body flange. There is a hole in the center of the flange which surrounds the stoma. There is a donut shaped soft gasket that surrounds the stoma and sticks and seals to the skin of the person using it. The present physical structure of this bag is a bag that has two openings. One opening has a plastic, snap-on attachment ring, surrounding the opening, used to snap and seal onto the body flange mentioned above. The second opening is held closed with a clip until the stoma waste needs to be emptied (when the bag fills or is nearly full). This second opening allows the waste material to exit the bag. This bag is called an ostomy bag.

The problem with present ostomy collection systems as the one described in paragraph 001 above is that when the ostomy bag becomes full of human waste, there is nowhere for the stoma waste to go when the pressure inside the bag reaches a critical amount. Usually when the pressure exceeds this amount, the stoma waste is forced out of the bag around the stoma onto the person's skin by being forced under the gasket which seals the body flange to the skin surrounding the stoma. When this happens, human waste is spread out onto the skin and clothes of the person wearing it. Once the seal of this ring is broken, there is no way to stop the spread of body waste exiting the bag. The sealing ring at this point becomes ineffective, and the body flange and seal must be replaced.

Another problem with the present art is that the ostomy bag can also be bumped or squeezed when a person is sleeping and rolls over on it, or it can be squeezed when the bag is only partially full by bumping against an object such as piece of furniture. When this happens, the stoma waste can be forced out again as described in paragraph 002 and can soak onto the person's clothes or the bed. This is very embarrassing and unsanitary for the person using this device.

The invention described here prevents the bag from breaking through the stoma ring gasket by allowing the excess stoma waste to be automatically transferred to a second, backup, ostomy bag through a one way valve which opens in the event that the pressure of the stoma waste in the first, primary, ostomy bag reaches a level just under the pressure that would allow the stoma waste to break the body flange seal and spill out onto the person wearing the ostomy bag.

This invention also has the advantages of having the ability to use present ostomy stoma waste collection bags presently on the market for the secondary backup bag, thus making the invention easy to adapt to an ileostomy patient's existing medical supplies, and method of usage.

### BRIEF SUMMARY OF THE INVENTION

This new invention is a device and a method by which stoma waste (from a person who has had an Ileostomy) can safely and securely overflow into a second ostomy bag should the first or primary ostomy bag become too full to handle any more intake of stoma waste, or in a situation where the first or primary ostomy bag becomes physically bumped or should happen to be squeezed to such an extent that the stoma waste from the primary bag would otherwise break through the seal of the ostomy body flange and onto the person wearing it.

Present day ostomy bags have two openings. One opening, which we will call the first opening for this discussion, has a connecting ring which allows it to connect and seal to a common, present art, ostomy body flange for the purpose of collecting human waste from the person's stoma. Present day ostomy bags also have another opening, which we will call the second opening for discussion purposes here, which is used for the purpose of emptying the stoma waste into a common toilet when the bag becomes full.

The new invention is an ostomy stoma waste collection bag, as described above, with the exception of an additional, or third, opening in the bag having an attachment ring of opposite gender, but similar to the first opening and having the ability to attach and seal to a second, present art style, ostomy bag having a similar style connecting ring.

This invention also consists of a one way fluid flow check valve sealed within this new, third, ostomy bag opening. The valve is connected in such a way as to have the direction of flow such as to allow fluid to flow out of this new, three opening, ostomy bag, and into a second or backup ostomy bag. This new bag having two connecting ring openings may be made of any shape or size made so it will be comfortable for a person who has had an ileostomy to wear it. Each person has a different size and shape, and may have a stoma located in such a place as to have one shaped device more comfortable then another.

### BRIEF DESCRIPTION OF THE SEVERAL VIEWS OF THE DRAWINGS

FIG. 1 shows the invention. Ref A, FIG. 1 shows an ostomy bag made of a material similar to present art ostomy bags. Ref B, FIG. 1 is the first, or primary, opening in the bag which is similar to a present art ostomy bag opening. Ref C, FIG. 1 is a connecting ring similar to present art ostomy bag connecting rings. This ring is used, as in present art ostomy bags, to connect and seal to a present art ostomy body flange. The body flange sticks to a person who has had an ileostomy, and is used to connect to an ostomy bag for the purpose of collecting human body waste which flows from the person's stoma, or opening in their abdomen. The ostomy bag is needed to store the stoma waste until the person using it empties the bag into a common toilet. Ref M, FIG. 1 is an opening in the bag, as is common in present art ostomy bags, and is needed to allow the stoma waste to empty from the bag. Ref F, FIG. 1 is a one-way check valve and is connected and sealed through a third opening in the ostomy bag. Check valves are very common and exist in the market today in many various sizes and shapes. The check valve allows fluid to flow in one direction through the check valve when the pressure of the fluid exceeds the cracking pressure of the check valve. Check valves are made today with many various cracking, or opening, pressures. Fluid will not flow through a check valve in the opposite direction. Ref E, FIG. 1 shows the skin material of the ostomy bag and is not an opening in the bag surrounding the check valve. Ref D, FIG. 1 is a connecting ring, similar to the connecting ring in Ref C, FIG. 1 except of opposite connecting gender. This ring is used to connect and seal to a second ostomy bag that is a standard ostomy bag as in the present art, and commonly found in today's market. The second ostomy bag is not shown in FIG. 1.

FIG. 2 shows the invention, the new ostomy bag in Ref E, FIG. 2, connected to a common present art ostomy bag shown in Ref F, FIG. 2 via the attached and sealed check valve shown in Ref K, FIG. 2. Ref L, FIG. 2 shows a connecting device, or ring, which connects and seals the check valve, Ref K, FIG. 2, to the first or primary ostomy bag. Ref J, FIG. 2 shows a connecting device or ring connected and sealed to a second ostomy bag, Ref F, FIG. 2.

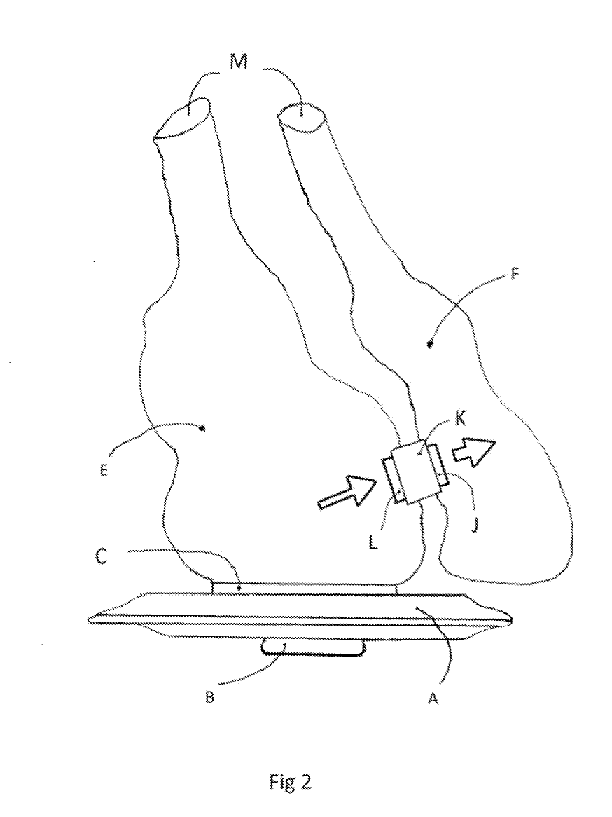

Ref A, FIG. 2 shows a common, present art, ostomy body flange. Ref B, FIG. 2 shows a present art ostomy ring seal. This seal is used to help attach and seal the flange unit to the skin of the person using it. Ref C, FIG. 2 is a present art, connecting ring used to connect and seal the primary ostomy bag to the ostomy body flange.

### DETAILED DESCRIPTION OF THE INVENTION

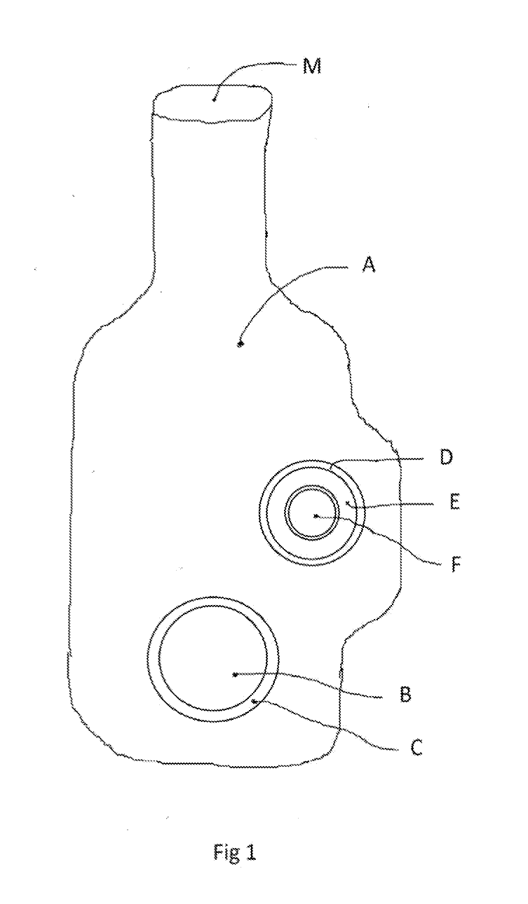

A present art ostomy bag has only two openings. One opening is used to connect to the present art ostomy body flange by a plastic ring connector. The second opening is a basic opening which is used to empty the stoma body waste into a toilet. This opening is held dosed with a present art plastic dipping device. This invention, shown in Ref A, FIG. 1, is a similar ostomy bag with a similar opening, Ref B, FIG. 1, connecting and sealing ring, Ref C, FIG. 1, such as described above which allows it to attach and seal to a present art ostomy body flange, shown in Ref A, FIG. 2. There is a second opening in this primary bag, shown in Ref M, FIG. 1, which allows the waste to be emptied as in existing ostomy present art bags today. This invention has a third opening which has a one way check valve attached and sealed to the bag. Ref F, FIG. 1 shows the check valve connected and sealed to the ostomy bag, Ref A, FIG. 1. The surrounding skin of the ostomy bag is shown around the check valve in Ref E, FIG. 1. Ref D, FIG. 1 shows a similar connecting and sealing ring which is of opposite gender of the ring shown in Ref C, FIG. 1. The purpose of this ring is to connect to a second ostomy bag as shown in Ref F, FIG. 2.

FIG. 2 shows how the invention is used. Ref A, FIG. 2 is a common ostomy body flange used today. The flange is sealed to the ileostomy patient's skin with a sticky ring seal shown in Ref B, FIG. 2. The invention is a three opening ostomy bag, shown in Ref E, FIG. 2. This bag is attached to the body flange, Ref A, FIG. 2, with a common, present art, plastic ring connector shown in Ref C, FIG. 2. The improvement here over present art ostomy collecting systems and bags is the third opening having the one way check valve, Ref K, FIG. 2, which is connected and sealed between the primary bag, Ref E, FIG. 2, and the secondary ostomy bag shown in Ref F, FIG. 2. The connecting device can be the same style as the present day ostomy connectors as Ref C, FIG. 2, or they can be other styles, or may be connected by permanent connecting and sealing methods such as glue, or melting of the skin of the ostomy bag to the valve. The connecting points are shown in Ref L, FIG. 2 for the inlet side of the check valve, and in Ref J, FIG. 2 at the outlet side of the valve. Both emptying openings of both ostomy bags are shown in Ref M, FIG. 2. These are held closed with standard ostomy clamping units as are used in present art ostomy supplies.

The operation of the invention is as follows. The first, or primary, ostomy bag fills through the normal process of the human body releasing human waste from the person's stoma. This waste flows through the body flange, Ref A, FIG. 2, and is prevented from leaking onto the person wearing it by the sealing ring shown in Ref B, FIG. 2. The waste material flows into the primary ostomy bag, Ref E, FIG. 2, and begins to fill the bag over time. The waste material cannot flow into the secondary bag at this time because under normal pressures, the valve will not open. The primary bag continues to fill until the bag is filled and begins to increase in pressure within the bag. If the person wearing the bag does not empty it because this person is too busy, or if the bag is bumped, the pressure in the bag would increase to a point where the ring seal, Ref B, FIG. 2, would no longer be able to function as a sealing agent. When this happens, as in present art systems, the human waste would break out of the seal and spill onto the person wearing it. The waste would also stain their clothes, or the bed they are sleeping in. With this invention, the spill would be prevented. As the pressure builds, it will reach a point where the check valve would open before the critical seal pressure would be reached. The opening pressure of the valve, Ref K, FIG. 2, would be a fixed value, set at a lower pressure then the breaking pressure of the stoma sticky seal, Ref B, FIG. 2. When the pressure in the bag reaches the opening pressure of the valve, the valve would open and allow the stoma waste to move from the primary bag to the backup, secondary, bag, Ref F, FIG. 2. This would prevent the waste from spilling onto the person and the person's clothes and bed. The waste in the secondary bag, Ref F, FIG. 2, cannot move back into the first bag, Ref E, FIG. 2, because the valve only flows in one direction. Once the pressure drops, after releasing pressure and fluid into the second bag, the valve automatically closes again. The person wearing the second bag will not feel the burden of carrying this second bag because during the normal operating pressures, the secondary bag would be flat. This new system allows the person more time to empty the bags in the event that they are unable to empty the primary bag earlier, and also allows the person wearing an ostomy device to feel more secure. When they do empty the bags, the clips cab be removed, the bags emptied, and then both bags can be reused. Under present art systems, when a seal breaks, the flange, seal and bag has to be replaced as well as having an unsanitary mess to clean up. The arrows in FIG. 2 show the direction of waste flow when the valve opens, should the bag overfill or be bumped or squeezed. When overfilling or bumping of the ostomy bag occurs, the stoma waste pressure will rise within this closed system. The opening of the one way valve occurs at a preset pressure which would be under the pressure needed to break the ostomy body flange seal. The breaking of this seal (which is the seal between the skin of an ileostomy patient and the ostomy body flange) would cause human waste to be released onto the person and the clothes of the person, or also to the bed sheets and blankets of the person's bed. This is unsanitary and very distressful for the person wearing such a device. Present art systems have no way of channeling the body waste to a secure and sanitary storage place. When this valve opens, the excess stoma waste can flow to the secondary or backup ostomy bag. When the pressure drops, the valve automatically closes and the waste in the primary bag, Ref E, FIG. 2, cannot flow into the second bag, Ref F, FIG. 2. This pressure release allows the user time to empty the bags through the standard present way by taking a plastic clip off of the empty openings of the bags, and emptying both bags into a common toilet, as the person usually would.

What is claimed in this invention is:

1. An improvement over a present art ostomy stoma waste collection bag, by which the collection ostomy bag will now have the improvement of an additional, or third, opening in this ostomy bag, which said opening has the inlet side of a one way flow check valve sealed within this third opening such that the overfilling of stoma body waste within this bag, or the squeezing of this bag would open the one way valve at some fixed pressure so the stoma waste material would be allowed to flow out of this bag through this valve and release the excess pressure and stoma waste into a second bag, sealed and secures to the outlet side of the check valve. The fixed pressure of this valve would be set at a pressure lower than the pressure that would break the seal that attaches the ostomy body flange to the person wearing the device.

2. The process by which the ostomy bag described in claim 1 can release human stoma waste, and route the excess waste material to a second ostomy bag via a one way flow check valve, when the primary bag becomes too full to collect any more waste material, or becomes bumped so as to cause the pressure in the first ostomy bag to rise to a point just below the pressure that would cause the waste material to break through the seal of an ostomy body flange, attached to a person who has had an ileostomy.
